(19) United States
(12) Patent Application Publication
(10) Pub. No.: US 2025/0049754 A1
(43) Pub. Date: Feb. 13, 2025

(54) COMPOSITIONS COMPRISING UROLITHIN FOR TREATING MUSCLE DECLINE AND A KIDNEY DYSFUNCTION

(71) Applicant: SOCIETE DES PRODUITS NESTLE S.A., Vevey (CH)

(72) Inventors: VINCENZO SORRENTINO, Lausanne (CH); SONIA KARAZ, Epalinges (CH); MAXIMILIAN VON EYNATTEN, La Conversion (CH); CLAUDIA ROESSLE, Morges (CH); JEROME FEIGE, Crissier (CH)

(21) Appl. No.: 18/720,716

(22) PCT Filed: Dec. 15, 2022
(86) PCT No.: PCT/EP2022/086049

§ 371 Date: Jun. 17, 2024

Related U.S. Application Data
(60) Provisional application No. 63/265,711, filed on Dec. 20, 2021.
(60) Provisional application No. 63/354,317, filed on Jun. 22, 2022.

(51) Int. Cl. A61K 31/37; A61P 13/12

(52) U.S. Cl. CPC A61K 31/37; A61P 13/12

(57) ABSTRACT

The present disclosure generally relates to methods and compositions comprising urolithin that treat or prevent a disease or condition associated with muscle decline and/or a kidney dysfunction. Moreover, methods and compositions comprising urolithin for increasing the urinary creatinine and/or for decreasing the albumin/creatinine ratio and/or for increasing the albumin reabsorption. In addition, methods and compositions comprising urolithin to improve the lean mass of a muscle and/or to improve the muscle fiber size and/or to improve the level of at least one amino acid are disclosed. Also disclosed are methods and compositions comprising urolithin to improve the level of at least one metabolic product and/or to improve the level of at least one nucleotide and/or to improve the level of at least one nicotinamide adenine dinucleotide and/or to increase the level of glutathione (GSH) and/or glutathione disulfide (GSSG) and/or to increase the level of succinate and/or malate and/or to increase the level of phosphocreatine.

The present disclosure generally relates to methods and compositions comprising urolithin that treat or prevent a disease or condition associated with muscle decline and/or a kidney dysfunction. Moreover, methods and compositions comprising urolithin for increasing the urinary creatinine and/or for decreasing the albumin/creatinine ratio and/or for increasing the albumin reabsorption. In addition, methods and compositions comprising urolithin to improve the lean mass of a muscle and/or to improve the muscle fiber size and/or to improve the level of at least one amino acid are disclosed. Also disclosed are methods and compositions comprising urolithin to improve the level of at least one metabolic product and/or to improve the level of at least one nucleotide and/or to improve the level of at least one nicotinamide adenine dinucleotide and/or to increase the level of glutathione (GSH) and/or glutathione disulfide (GSSG) and/or to increase the level of succinate and/or malate and/or to increase the level of phosphocreatine.

### BACKGROUND

The loss of muscle mass that occurs during muscle wasting may be characterized by muscle protein degradation due to catabolism. Muscle protein catabolism, whether caused by a high degree of protein degradation or a low degree of protein synthesis, leads to a decrease in muscle mass and to muscle wasting.

Muscle decline in form of muscle wasting is associated with chronic, neurological, genetic or infectious pathologies, diseases, illnesses or conditions. In particular muscle decline is related to chronic kidney disease (CKD), end stage renal failure (ESRD), metabolic dysfunction-induced muscle wasting, dialysis, diabetes, muscle loss and/or kidney failure due to hospitalization in the intensive care unit, Pompe disease, metabolic acidosis, methylmalonic aciduria, disuse atrophy, protein-energy wasting amongst others.

Muscle wasting is a common complication of CKD, characterized by the loss of muscle mass, strength and function, which significantly increases the risk of morbidity and mortality in this population. Numerous complications associated with declining renal function and lifestyle activate catabolic pathways and impair muscle regeneration, resulting in substantial protein wasting.

CKD is a gradual and progressive loss of the ability of the kidneys to excrete wastes, concentrate urine, reabsorb proteins and amino acids, and conserve electrolytes. Unlike acute kidney failure with its abrupt but reversible of kidney function, the kidney functions in chronic kidney disease progress and deteriorate irreversibly towards end stage renal disease (ESRD). CKD arises from many heterogeneous disease pathways that alter the function and structure of the kidney irreversibly, over months or years.

Diabetes and hypertension are the main causes of CKD in all high-income and middle-income countries, and also in many low-income countries. Incidence, prevalence, and progression of CKD also vary within countries by ethnicity and social determinants of health, possibly through epigenetic influence. Many people are asymptomatic or have non-specific symptoms such as lethargy, itch, or loss of appetite. Diagnosis is commonly made after chance findings from screening tests such as urinary dipstick or blood tests, or when symptoms become severe. The best available indicator of overall kidney function is the glomerular filtration rate (GFR). Disease and management are classified according to stages of disease severity, which are assessed from GFR and albuminuria, and clinical diagnosis (cause and pathology). Presence of proteinuria is associated with increased risk of progression of CKD. The diagnosis of CKD rests on establishing a chronic reduction in kidney function and structural kidney damage.

CKD is diagnosed using a staging system that demonstrates the amount of kidney function available (stage 1=normal kidney function) and patients often do not present symptoms in the early stages. Stage 5 of CKD is ESRD, which is a complete or near complete failure of the kidneys and usually occurs when kidney function is less than 10% of baseline.

### SUMMARY OF THE INVENTION

In a first aspect, the present disclosure provides a method of treating and/or preventing a disease or condition associated with muscle decline and/or a kidney dysfunction. The method comprises administering to a subject in need thereof an effective amount of a composition comprising urolithin or an urolithin or a pharmaceutically acceptable salt thereof. In one embodiment the muscle decline and/or the kidney dysfunction is treated and/or prevented in an early stage of chronic kidney disease and/or a late stage of chronic kidney disease.

In a second aspect, the present disclosure provides a method for improving the lean mass of a muscle in a subject in need thereof, the method comprising administering to the subject in need thereof an effective amount of a composition comprising urolithin or an urolithin or a pharmaceutically acceptable salt thereof. In one embodiment, the lean mass of a muscle is improved in an early stage of chronic kidney disease and/or a late stage of chronic kidney disease.

In some embodiments of the aforementioned aspects, the muscle is tibialis anterior and/or quadriceps.

In a third aspect, the present disclosure provides a method for increasing the albumin reabsorption in a subject in need thereof. The method comprises administering to the subject in need thereof an effective amount of a composition comprising urolithin or an urolithin or a pharmaceutically acceptable salt thereof. In one embodiment, the albumin reabsorption is increased in an early stage of chronic kidney disease and/or a late stage of chronic kidney disease.

In a fourth aspect, the present disclosure provides a method for increasing the urinary creatinine in a subject in need thereof. The method comprises administering to the subject in need thereof an effective amount of a composition comprising urolithin or an urolithin or a pharmaceutically acceptable salt thereof. In one embodiment, the urinary creatinine is increased in an early stage of chronic kidney disease and/or a late stage of chronic kidney disease.

In a fifth aspect, the present disclosure provides a method for decreasing the albumin/creatinine ratio in a subject in need thereof. The method comprises administering to the subject in need thereof an effective amount of a composition comprising urolithin or an urolithin or a pharmaceutically acceptable salt thereof. In one embodiment, the albumin/creatinine ratio is decreased in an early stage of chronic kidney disease and/or a late stage of chronic kidney disease.

In a sixth aspect, the present disclosure provides a method for increasing the urinary carnosine in a subject in need thereof. The method comprises administering to the subject in need thereof an effective amount of a composition comprising urolithin or an urolithin or a pharmaceutically acceptable salt thereof. In one embodiment, the urinary carnosine is increased in an early stage of chronic kidney disease and/or a late stage of chronic kidney disease.

In a seventh aspect, the present disclosure provides a method for increasing the urinary anserine in a subject in need thereof. The method comprises administering to the subject in need thereof an effective amount of a composition comprising urolithin or an urolithin or a pharmaceutically acceptable salt thereof. In one embodiment, the urinary anserine is increased in an early stage of chronic kidney disease and/or a late stage of chronic kidney disease.

In an eight aspect, the present disclosure provides a method for increasing the urinary S-adenosylmethionine in a subject in need thereof. The method comprises administering to the subject in need thereof an effective amount of a composition comprising urolithin or an urolithin or a pharmaceutically acceptable salt thereof. In one embodiment, the urinary S-adenosylmethionine is increased in an early stage of chronic kidney disease and/or a late stage of chronic kidney disease.

In a ninth aspect, the present disclosure provides a method for improving the bone femur mass in a subject in need thereof. The method comprises administering to the subject in need thereof an effective amount of a composition comprising urolithin or an urolithin or a pharmaceutically acceptable salt thereof. In one embodiment, the bone femur mass is improved in an early stage of chronic kidney disease and/or a late stage of chronic kidney disease.

In some embodiments of the aforementioned aspects, the muscle is tibialis anterior and/or quadriceps.

In a tenth aspect, the present disclosure provides a method for improving the muscle fiber size in a subject in need thereof, the method comprising administering to the subject in need thereof an effective amount of a composition comprising urolithin or an urolithin or a pharmaceutically acceptable salt thereof.

In an eleventh aspect, the present disclosure provides a method for improving the level of at least one amino acid in a muscle of a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the level of at least one amino acid in a muscle of a subject in need thereof.

In a twelfth aspect, the present disclosure provides a method for improving the level of at least one metabolic product in a muscle of a subject in need thereof, the method comprising administering to the subject in need thereof an effective amount of a composition comprising urolithin or an urolithin or a pharmaceutically acceptable salt thereof.

In a thirteenth aspect, the present disclosure provides a method for improving the level of at least one nucleotide in a muscle of a subject in need thereof, the method comprising administering to the subject in need thereof an effective amount of a composition comprising urolithin or an urolithin or a pharmaceutically acceptable salt thereof.

In a fourteenth aspect, the present disclosure provides a method for improving the level of at least one nicotinamide adenine dinucleotide in a muscle of a subject in need thereof, the method comprising administering to the subject in need thereof an effective amount of a composition comprising urolithin or an urolithin or a pharmaceutically acceptable salt thereof.

In a fifteenth aspect, the present disclosure provides a method for increasing the level of glutathione (GSH) and/or glutathione disulfide (GSSG) in a muscle of a subject in need thereof, the method comprising administering to the subject in need thereof an effective amount of a composition comprising urolithin or an urolithin or a pharmaceutically acceptable salt thereof.

In a sixteenth aspect, the present disclosure provides a method for increasing the level of succinate and/or malate in a muscle of a subject in need thereof, the method comprising administering to the subject in need thereof an effective amount of a composition comprising urolithin or an urolithin or a pharmaceutically acceptable salt thereof.

In a seventeenth aspect, the present disclosure provides a method for increasing the level of phosphocreatine in a muscle of a subject in need thereof, the method comprising administering to the subject in need thereof an effective amount of a composition comprising urolithin or an urolithin or a pharmaceutically acceptable salt thereof.

In some embodiments of the aforementioned aspects, the subject has a disease or condition selected from chronic kidney disease, metabolic induced muscle wasting, end-stage renal disease, dialysis, diabetes, muscle loss and/or kidney failure due to hospitalization in the intensive care unit, Pompe disease, metabolic acidosis, methylmalonic aciduria, disuse atrophy, protein-energy wasting and combinations thereof.

In other embodiments, the composition comprising urolithin or an urolithin or a pharmaceutically acceptable salt thereof is administered enterally.

In further embodiments, the composition comprising urolithin or an urolithin or a pharmaceutically acceptable salt thereof is administered parenterally.

In some embodiments, the urolithin is micronized.

In other embodiments, the urolithin is urolithin A.

In an eighteenth aspect, the present disclosure provides a nutritional composition for use in treating and/or preventing a disease or condition associated with muscle decline and/or a kidney dysfunction. The nutritional composition comprises a composition comprising urolithin or an urolithin or a pharmaceutically acceptable salt thereof. Preferably the nutritional composition contains an amount of urolithin effective for treating and/or preventing a disease or condition associated with muscle decline and/or a kidney dysfunction in a subject in need thereof.

In one embodiment, the composition is selected from the group consisting of a food product, a food for special medical purposes (FSMP), a nutritional supplement, a dairy-based drink, a low-volume liquid supplement, a meal replacement beverage and combinations thereof.

In another embodiment, the composition is formulated for oral administration.

In a nineteenth aspect, the present disclosure provides a unit dosage form for use in a method of treating and/or preventing a disease or condition associated with muscle decline and/or a kidney dysfunction. The unit dosage form comprises a composition comprising urolithin or an urolithin or a pharmaceutically acceptable salt thereof. Preferably, the unit dosage form contains an amount of urolithin effective for treating and/or preventing a disease or condition associated with muscle decline and/or a kidney dysfunction in a subject in need thereof.

In one embodiment, the unit dosage form is selected from the group consisting of a food product, a food for special medical purposes (FSMP), a nutritional supplement, a dairy-based drink, a low-volume liquid supplement, a meal replacement beverage and combinations thereof.

In another embodiment, the unit dosage form is formulated for enteral administration.

In some embodiments of the eighteenth and nineteenth aspect, the composition and/or the unit dosage form contains an amount of urolithin effective to increase the urinary creatinine and/or to decrease the albumin/creatinine ratio and/or to increase the albumin reabsorption and/or to improve the lean mass of a muscle and/or to increase the urinary carnosine and/or to increase the urinary anserine and/or to increase urinary S-adenosylmethionine and/or to improve the bone femur mass.

In other embodiments of the eighteenth and nineteenth aspect, the amount of the composition comprising urolithin or the urolithin or a pharmaceutically acceptable salt thereof is effective to improve the lean mass of a muscle and/or to improve the muscle fiber size and/or to improve the level of at least one amino acid.

In further embodiments of the eighteenth and nineteenth aspect, the amount of the composition comprising urolithin or the urolithin or a pharmaceutically acceptable salt thereof is effective to improve the level of at least one metabolic product and/or to improve the level of at least one nucleotide and/or to improve the level of at least one nicotinamide adenine dinucleotide and/or to increase the level of glutathione (GSH) and/or glutathione disulfide (GSSG) and/or to increase the level of succinate and/or malate and/or to increase the level of phosphocreatine.

An advantage of one or more aspects and embodiments provided by the present disclosure is to help maintain healthy muscle mass.

Another advantage of one or more aspects and embodiments provided by the present disclosure is to help stabilizing a kidney dysfunction.

Yet another advantage of one or more aspects and embodiments provided by the present disclosure is to help ameliorating kidney function.

An advantage of one or more aspects and embodiments provided by the present disclosure is to help improving the lean mass of a muscle.

Another advantage of one or more aspects and embodiments provided by the present disclosure is to help increasing the albumin reabsorption.

Yet another advantage of one or more aspects and embodiments provided by the present disclosure is to help increasing the urinary creatinine.

An advantage of one or more aspects and embodiments provided by the present disclosure is to help decreasing the albumin/creatinine ratio.

Another advantage of one or more aspects and embodiments provided by the present disclosure is to help increasing urinary carnosine.

Yet another advantage of one or more aspects and embodiments provided by the present disclosure is to help increasing urinary anserine.

An advantage of one or more aspects and embodiments provided by the present disclosure is to help increasing urinary S-adenosylmethionine.

Another advantage of one or more aspects and embodiments provided by the present disclosure is to help improving the bone femur mass.

An advantage of one or more aspects and embodiments provided by the present disclosure is to improve the endurance and/or efficiency of a muscle.

Another advantage of one or more aspects and embodiments provided by the present disclosure is to improve the muscle fiber size

Yet another advantage of one or more aspects and embodiments provided by the present disclosure is to improve the amino acid level in a muscle.

An advantage of one or more aspects and embodiments provided by the present disclosure is to reduce muscle atrophy.

Another advantage of one or more aspects and embodiments provided by the present disclosure is to increase the muscle bioenergetics nucleotides and nucleotides important for cellular signaling.

Additional features and advantages are described herein and will be apparent from the following Figures and Detailed Description.

### BRIEF DESCRIPTION OF THE FIGURES

FIG. 1a shows the albumin/creatine ratio measured in mice after 5 weeks (Midpoint) treatment with Urolithin A, dosed at 50 mg/kg/day (+UA, db/db+UA group) compared to a vehicle group (Diabetic, db/db+V group) and a control group (Control, db/m+V group) after 5 weeks treatment with 100 μl of 0.5% carboxymethylcellulose. Creatinine was determined using a Cobas Integra autoanalyzer. Excretion of urine albumin was measured by ELISA kit.

FIG. 1b shows the albumin/creatine ratio measured in mice after 10 weeks (Endpoint) treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose. Creatinine was determined using a Cobas Integra autoanalyzer. Excretion of urine albumin was measured by ELISA kit.

FIG. 2 shows the urinary creatinine measured in mice after 10 weeks treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose. Urinary creatinine was determined using a Cobas Integra autoanalyzer.

FIG. 3 shows the progression of albuminuria measured in mice at baseline (time zero), after 5 weeks (Midpoint) and after 10 weeks (Endpoint) treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose. Excretion of urine albumin was measured by ELISA kit.

FIG. 4 shows the lean mass of muscles in grams measured in mice after 10 weeks treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose. The lean mass of muscles was measured using EchoMRI.

FIG. 5a shows the muscle mass of the tibialis anterior in milligrams measured in mice after 10 weeks treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose. The muscle mass of the tibialis anterior was measured by weighing on a precision scale.

FIG. 5b shows the muscle mass of the quadriceps in milligrams measured in mice after 10 weeks treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose. The muscle mass of the quadriceps was measured by weighing on a precision scale.

FIG. 6 shows the urinary carnosine measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. Urinary carnosine was determined in a semi-quantitative manner using cold methanol:water:chloroform (5:3:5 (v/v)) extraction to separate the polar metabolites and apolar metabolites. In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH4OH), pH ˜9.3, and solvent B was acetonitrile (ACN). The eluting metabolites, including carnosine, were analyzed with an orbitrap mass spectrometer (Orbitrap Fusion Lumos Tribrid, Thermo Scientific) equipped with a heated electrospray ionisation (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing

FIG. 7 shows the urinary anserine measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. Urinary anserine was determined in a semi-quantitative manner using cold methanol:water:chloroform (5:3:5 (v/v)) extraction to separate the polar metabolites and apolar metabolites. In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH4OH), pH ˜9.3, and solvent B was acetonitrile (ACN). The eluting metabolites, including anserine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 8 shows the urinary S-adenosylmethionine measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. Urinary S-adenosylmethionine was determined in a semi-quantitative manner using cold methanol:water:chloroform (5:3:5 (v/v)) extraction to separate the polar metabolites and apolar metabolites. In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH4OH), pH ˜9.3, and solvent B was acetonitrile (ACN). The eluting metabolites, including S-adenosylmethionine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 9 shows the bone femur mass measured in mice after 10 weeks treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose. The bone femur mass was determined by weighing on a precision scale.

FIG. 10 shows the fiber size distribution measured on tibilais interior in mice after 10 weeks treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose. The fiber size distribution was determined after tibialis anterior cryosection, stained for the laminin protein and the myo-nucleus. All slides were acquired with the Olympus VS120 slide scanner microscope. The size of myofibers was calculated with Min Feret using an automated image processing algorithm developed internally using QuPath software and Fiji's tool open-CSAM.

FIG. 11a shows the isoleucine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The isoleucine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including isoleucine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 11b shows the methionine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The methionine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including methionine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 11c shows the lysine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The lysine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including lysine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 11d shows the tyrosine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The tyrosine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH4Ac) and 0.04% (v/v) ammonium hydroxide (NH₄OH), pH ˜9.3, and solvent B was acetonitrile (ACN). The eluting metabolites, including tyrosine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 11e shows the proline level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The proline level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including proline, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 11f shows the alanine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The alanine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including alanine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 11g shows the glycine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The fiber size distribution was determined The glycine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including glycine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 12a shows the N-acetyl-DL-serine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The N-acetyl-DL-serine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including N-acetyl-DL-serine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 12b shows the N-acetyl-L-arginine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The N-acetyl-L-arginine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including N-acetyl-L-arginine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 12c shows the N-acetylglutamic acid level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The N-acetylglutamic acid level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including N-acetylglutamic acid, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 13a shows the N,N-dimethylglycine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The N,N-dimethylglycine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including N,N-dimethylglycine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 13b shows the S-adenosylmethionine (SAM) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The N S-adenosylmethionine (SAM) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including S-adenosylmethionine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 14a shows the 2-hydroxybutyrate level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The 2-hydroxybutyrate level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including 2-hydroxybutyrate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 14b shows the trans-urocanic acid level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The trans-urocanic acid was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including trans-urocanic acid, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 15a shows the muscular anserine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular anserine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular anserine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 15b shows the muscular carnosine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular carnosine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular carnosine level, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 16a shows the muscular adenosine triphosphate (ATP) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular adenosine triphosphate (ATP) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including adenosine triphosphate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 16b shows the muscular adenosine diphosphate (ADP) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular adenosine diphosphate (ADP) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular adenosine diphosphate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 16c shows the muscular guanosine-triphosphate (GTP)level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular guanosine-triphosphate (GTP) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular guanosine-triphosphate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 16d shows the muscular guanosine-diphosphate (GDP) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular guanosine-diphosphate (GDP) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular guanosine-diphosphate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 16e shows the muscular uridine-triphosphate (UTP) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular uridine-triphosphate (UTP) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular uridine-triphosphate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 16f shows the muscular cytidine triphosphate (CTP) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular cytidine triphosphate (CTP) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular cytidine triphosphate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 16g shows the muscular flavin adenine dinucleotide (FAD) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular flavin adenine dinucleotide (FAD) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular flavin adenine dinucleotide, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 17a shows the muscular NAD+ level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular NAD+ level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including muscular NAD+, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 17b shows the muscular NADP+ level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular NADP+ level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including NADP+, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 18a shows the muscular glutathione (GSH) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular glutathione (GSH) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including GSH, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 18b shows the muscular glutathione disulfide (GSSG) level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular glutathione disulfide (GSSG) level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including GSSG, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 19a shows the muscular succinate level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular succinate level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including succinate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 19b shows the muscular malate level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular malate level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including malate, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

FIG. 20 shows the muscular phosphocreatine level measured in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose. The muscular phosphocreatine level was determined using a liquid-liquid extraction with ¹³C-yeast as internal standards. The muscle extraction were done with metal beads in pre-cooled racks (−80° C.) for 2 min at 23 Hz in a tissue mixer (Qiagen TissueLyser II). In addition, a protein layer remained in the middle between the two phases. The polar phase was dried overnight in a vacuum centrifuge at 4° C. and 5 mbar, and was dissolved in 60 μl 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H₂O with 10 mM ammonium acetate (NH₄Ac) and 0.04% (v/v) ammonium hydroxide (NH₄OH), pH ˜9.3, and solvent B was acetonitrile (ACN). The eluting metabolites, including phosphocreatine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified. A composition or dosage unit “consisting essentially of” contains at least 50 wt. % of the referenced components, preferably at least 75 wt. % of the referenced components, more preferably at least 85 wt. % of the referenced components, most preferably at least 95 wt. % of the referenced components.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “muscle decline and/or a kidney dysfunction” should be interpreted as “muscle decline” or “a kidney dysfunction” or “both muscle decline and a kidney dysfunction”.

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

A “subject” or “individual” is a mammal, preferably a human. The term mammal may include but is not limited to a pet or a farm animal. In particular the term “farm animal” may include but is not limited to a horse (e.g., a pet or horse undergoing medical treatment), or cattle or poultry (e.g., cattle or poultry being used in agriculture). In particular the term “pet” may include but is not limited to senior pets (e.g., cats aged above 10 years or dogs aged above 7 years), pets with obesity, pets with diabetes and pets with CKD.

As used herein, an “effective amount” is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced” and the like refer to the effects of the composition disclosed herein, namely a composition comprising urolithin or an urolithin or a pharmaceutically acceptable salt thereof.

The terms “food,” “food product” and “food composition” mean a product or composition that is intended for ingestion by an individual such as a human and provides at least one nutrient to the individual. A food product typically includes at least one of a protein, a lipid, a carbohydrate and optionally includes one or more vitamins and minerals. The compositions of the present disclosure, including the many embodiments described herein, can comprise, consist of, or consist essentially of the elements disclosed herein, as well as any additional or optional ingredients, components, or elements described herein or otherwise useful in a diet.

The term “unit dosage form”, as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds and/or compositions employed, the effect to be achieved, and the pharmacodynamics associated with each compound and/or composition in the host.

“Chronic kidney disease (CKD)” encompasses the presence of kidney damage (i.e., albuminuria) or decreased kidney function (i.e., GFR <60 mL/min per 1.73 m²) for 3 months or more, irrespective of clinical diagnosis. CKD is classified into five stages on the basis of GFR: more than 90 mL/min per 1.73 m² (stage 1), 60-89 mL/min per 1.73 m² (stage 2), 30-59 mL/min per 1.73 m² (stage 3), 15-29 mL/min per 1.73 m² (stage 4), and less than 15 mL/min per 1.73 m² (stage 5).

“Early stage of chronic kidney disease” encompasses chronic kidney disease in stages 2 and 3 on the basis of GFR: 60-89 mL/min per 1.73 m² (stage 2), 30-59 mL/min per 1.73 m² (stage 3). Preferably, “early stage of chronic kidney disease” encompasses chronic kidney disease in stage 2 on the basis of GFR: 60-89 mL/min per 1.73 m².

“Late stage of chronic kidney disease” encompasses chronic kidney disease in stages 4 and 5 on the basis of GFR: 15-29 mL/min per 1.73 m² (stage 4), and less than 15 mL/min per 1.73 m² (stage 5). Preferably, “late stage of chronic kidney disease” encompasses chronic kidney disease in stage 4 on the basis of GFR: 15-29 mL/min per 1.73 m².

“End-stage renal disease (ESRD)” encompasses the condition of individuals with CKD, who require a kidney replacement therapy. Preferably, ESRD encompasses chronic kidney disease in stage 5 on the basis of GFR: less than 15 mL/min per 1.73 m².

“Metabolic induced muscle wasting” encompasses a prolonged catabolic state, where muscle protein breakdown exceeds the rate of protein synthesis.

“Diabetes” encompasses both the type I and type II forms of the disease. Non-limiting examples of risk factors for diabetes include: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

“Pompe disease” encompasses the classic infantile form, non-classic infantile form, and late-onset form. In children with classic or non-classic infantile-onset Pompe disease, the activity of the acid alpha-glucosidase is generally less than about 1% of normal. In individuals with the late-onset form, the acid alpha-glucosidase is generally lower than about 40% of normal.

“Metabolic acidosis” encompasses a reduced serum pH, and an abnormal serum bicarbonate concentration of <22 mEq/L, below the normal range of 22 to 29 mEq/L.

“Methylmalonic aciduria” relates to an inherited disorder in which the body is unable to properly digest specific fats and proteins, and the amino acids methionine, threonine, isoleucine and valine; which in turn leads to a buildup of a toxic level of methylmalonic acid in the blood.

“Disuse atrophy” relates to a temporary condition if the unused muscles are exercised properly after a limb is taken out of a cast or a person has regained enough strength to exercise after being bedridden for a period of time.

“Protein-energy wasting” relates to a loss of body protein mass and fuel reserves in a subject due to a maladaptive metabolic state. The maladaptive metabolic state includes nonspecific inflammatory processes, transient, intercurrent catabolic illnesses; nutrient losses into dialysate, acidemia, endocrine disorders such as resistance to insulin, growth hormone, and insulin-like growth factor-1, hyperglucagonemia, hyperparathyroidism, and loss of blood into the hemodialyzer, into feces or by blood drawing.

“Metabolic product” or “metabolite” relates to an intermediate or end product of metabolism. The term “metabolic product” or “metabolite” may relate to an intermediate or end product of metabolism related to muscular metabolism and/or growth. In particular, the term “metabolic product” or “metabolite” may relate to an intermediate or end product of muscular metabolism.

### EMBODIMENTS

In a first aspect, the present disclosure provides a method of treating and/or preventing a disease or condition associated with muscle decline and/or a kidney dysfunction. The method comprises administering to a subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method of treating and/or preventing a disease or condition associated with muscle decline and/or a kidney dysfunction.

In one embodiment, the method is a method of treating at least one disease or condition associated with muscle decline and/or a kidney dysfunction. The method comprising administering to a subject having at least one disease or condition associated with muscle decline and/or a kidney dysfunction a composition comprising a prophylactically effective amount of a composition comprising urolithin or an urolithin or a pharmaceutically acceptable salt thereof.

In another embodiment, the method is a method of preventing at least one disease or condition associated with muscle decline and/or a kidney dysfunction. The method comprises administering to a subject at risk of the at least one disease or condition associated with muscle decline and/or a kidney dysfunction a composition comprising a prophylactically effective amount of a composition comprising urolithin or an urolithin or a pharmaceutically acceptable salt thereof.

In yet another embodiment, the muscle is tibialis anterior and/or quadriceps.

In one embodiment, the amount of the composition comprising urolithin or the urolithin or a pharmaceutically acceptable salt thereof is effective to increase the urinary creatinine and/or to decrease the albumin/creatinine ratio and/or to increase the albumin reabsorption and/or to improve the lean mass of a muscle and/or to increase the urinary carnosine and/or to increase the urinary anserine and/or to increase urinary S-adenosylmethionine and/or to improve the bone femur mass.

In a certain embodiment, the amount of the composition comprising urolithin or the urolithin or a pharmaceutically acceptable salt thereof is effective to increase the urinary creatinine and to improve the lean mass of a muscle.

In another embodiment, the amount of the composition comprising urolithin or the urolithin or a pharmaceutically acceptable salt thereof is effective to improve the lean mass of a muscle and/or to improve the muscle fiber size and/or to improve the level of at least one amino acid.

In a further embodiment the amount of the composition comprising urolithin or the urolithin or a pharmaceutically acceptable salt thereof is effective to improve the level of at least one metabolic product and/or to improve the level of at least one nucleotide and/or to improve the level of at least one nicotinamide adenine dinucleotide and/or to increase the level of glutathione (GSH) and/or glutathione disulfide (GSSG) and/or to increase the level of succinate and/or malate and/or to increase the level of phosphocreatine.

In a second aspect, the present disclosure provides a method for improving the lean mass of a muscle in a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the lean mass of a muscle in a subject in need thereof. In a related embodiment, the muscle is tibialis anterior and/or quadriceps.

In a third aspect, the present disclosure provides a method for increasing the albumin reabsorption in a subject in need thereof. The method comprises administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for increasing the albumin reabsorption in a subject in need thereof. Increasing the albumin reabsorption means that the amount of albumin is reduced in the urine.

In a fourth aspect, the present disclosure provides a method for increasing the urinary creatinine in a subject in need thereof. The method comprises administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for increasing the urinary creatinine in a subject in need thereof.

In a fifth aspect, the present disclosure provides a method for decreasing the albumin/creatinine ratio in a subject in need thereof. The method comprises administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for decreasing the albumin/creatinine ratio in a subject in need thereof.

In a sixth aspect, the present disclosure provides a method for increasing the urinary carnosine in a subject in need thereof. The method comprises administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for increasing the urinary carnosine in a subject in need thereof.

In a seventh aspect, the present disclosure provides a method for increasing the urinary anserine in a subject in need thereof. The method comprises administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for increasing the urinary anserine in a subject in need thereof.

In an eight aspect, the present disclosure provides a method for increasing the urinary S-adenosylmethionine in a subject in need thereof. The method comprises administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for increasing the urinary S-adenosylmethionine in a subject in need thereof.

In a ninth aspect, the present disclosure provides a method for improving the bone femur mass in a subject in need thereof. The method comprises administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the bone femur mass in a subject in need thereof.

The present disclosure provides a method for improving and/or maintaining the mass of a muscle in a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving and/or maintaining the mass of a muscle in a subject in need thereof. In particular, the amount of the composition comprising urolithin or the urolithin or a pharmaceutically acceptable salt thereof is effective to improve the lean mass of a muscle and/or to improve the muscle fiber size and/or to improve the level of at least one amino acid.

In a tenth aspect, the present disclosure provides a method for improving the muscle fiber size in a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the muscle fiber size in a subject in need thereof.

A decrease in muscle mass and fiber size may result in muscle atrophy. Thus, in one embodiment administering to the subject in need thereof an effective amount of a composition comprising urolithin or an urolithin or a pharmaceutically acceptable salt thereof reduces muscle atrophy.

In an eleventh aspect, the present disclosure provides a method for improving the level of at least one amino acid in a muscle of a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the level of at least one amino acid in a muscle of a subject in need thereof.

In one embodiment, the amino acid, the levels of which are increased are selected from the group consisting of isoleucine, methionine, lysine, tyrosine, proline, alanine and glycine. In one embodiment, the amino acid, the levels of which is increased is isoleucine and/or methionine and/or lysine and/or tyrosine and/or proline and/or alanine and/or glycine.

The present disclosure provides a method for improving the endurance and/or efficiency of a muscle in a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the endurance and/or efficiency of a muscle in a subject in need thereof. In particular, the amount of the composition comprising urolithin or the urolithin or a pharmaceutically acceptable salt thereof is effective to improve the level of at least one metabolic product and/or to improve the level of at least one nucleotide and/or to improve the level of at least one nicotinamide adenine dinucleotide and/or to increase the level of glutathione (GSH) and/or glutathione disulfide (GSSG) and/or to increase the level of succinate and/or malate and/or to increase the level of phosphocreatine.

The endurance and/or efficiency of a muscle may be improved by improving its metabolism.

Thus, in a twelfth aspect, the present disclosure provides a method for improving the level of at least one metabolic product in a muscle of a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the level of at least one metabolic product in a muscle of a subject in need thereof.

In any embodiment improving the level of metabolic products may also include increasing and/or deceasing the level of certain metabolites.

In one embodiment, the at least one metabolic product is selected from the group consisting of N,N-dimethylglycine, S-adenosylmethionine (SAM), N-acetyl-DL-serine, N-acetyl-L-arginine and N-acetylglutamic acid.

In another embodiment, the metabolic product is N-acetyl-DL-serine and/or N-acetyl-L-arginine and/or N-acetylglutamic acid.

In a further embodiment, the metabolic product is N,N-dimethylglycine and/or S-adenosylmethionine (SAM).

The method for improving the level of at least one metabolic product may also increase the level of ketone bodies and/or molecules related to ketone bodies. In one embodiment, the metabolite is 2-hydroxybutyrate.

In another embodiment, the level of at least one metabolic product is improved by decreasing the level of trans-urocanic acid.

In a further embodiment, the level of at least one metabolic product is improved by increasing the level of muscular anserine and/or carnosine.

Anserine and carnosine are related to muscle efficiency and endurance. In particular, carnosine serves as a physiological buffer, possesses antioxidant properties, influences enzyme regulation and affects sarcoplasmic reticulum calcium regulation. In one embodiment administering to the subject in need thereof an effective amount of a composition comprising urolithin or an urolithin or a pharmaceutically acceptable salt thereof improves the muscle efficiency and endurance.

In a thirteenth aspect, the present disclosure provides a method for improving the level of at least one nucleotide in a muscle of a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the level of at least one nucleotide in a muscle of a subject in need thereof.

In one embodiment, the at least one nucleotide is selected from the group consisting of adenosine triphosphate (ATP), adenosine diphosphate (ADP), guanosine-triphosphate (GTP), guanosine-diphosphate (GDP), uridine-triphosphate (UTP), cytidine triphosphate (CTP) and flavin adenine dinucleotide (FAD).

In another embodiment, the nucleotide is adenosine triphosphate (ATP) and/or adenosine diphosphate (ADP) and/or guanosine-triphosphate (GTP) and/or guanosine-diphosphate (GDP) and/or uridine-triphosphate (UTP) and/or cytidine triphosphate (CTP) and/or flavin adenine dinucleotide (FAD).

Nucleotides are a source of energy-biomolecules and bioenergetics. In addition, nucleotides are important for cellular signaling and participate for example in the purinergic signaling pathway. Thus, in a further embodiment, administering to the subject in need thereof an effective amount of a composition comprising urolithin or an urolithin or a pharmaceutically acceptable salt thereof increases the muscle bioenergetics nucleotides and nucleotides important for cellular signaling.

In a fourteenth aspect, the present disclosure provides a method for improving the level of at least one nicotinamide adenine dinucleotide in a muscle of a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for improving the level of at least one nicotinamide adenine dinucleotide in a muscle of a subject in need thereof.

In one embodiment, the nicotinamide adenine dinucleotide is nicotinamide adenine dinucleotide in its oxidized form (NAD+) or reduced form (NADH). Preferably, the adenine dinucleotide is nicotinamide adenine dinucleotide in its oxidized form (NAD+).

In another embodiment, the nicotinamide adenine dinucleotide is nicotinamide adenine dinucleotide phosphate (NADP). In particular, the nicotinamide adenine dinucleotide is nicotinamide adenine dinucleotide phosphate in its oxidized form (NADP+) or reduced form (NADPH). Preferably, the adenine dinucleotide is nicotinamide adenine dinucleotide phosphate in its oxidized form (NAD+).

In a fifteenth aspect, the present disclosure provides a method for increasing the level of glutathione (GSH) and/or glutathione disulfide (GSSG) in a muscle of a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for increasing the level of glutathione (GSH) and/or glutathione disulfide (GSSG) in a muscle of a subject in need thereof.

In one embodiment the level of glutathione (GSH) is increased. In another embodiment, the level of glutathione disulfide (GSSG) is increased.

In one embodiment, administering to the subject in need thereof an effective amount of a composition comprising urolithin or an urolithin or a pharmaceutically acceptable salt thereof improves the antioxidant buffering capacity of the muscle.

In a sixteenth aspect, the present disclosure provides a method for increasing the level of succinate and/or malate in a muscle of a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for increasing the level of succinate and/or malate in a muscle of a subject in need thereof.

In one embodiment, the level of succinate is increased in the muscle. In another embodiment, the level of malate is increased in the muscle.

Succinate and malate are metabolites of the tricarboxylic acid cycle (TCA), also known as citrate cycle. Metabolites of the TCA are important for bioenergetics. Thus, in one embodiment, administering to the subject in need thereof an effective amount of a composition comprising urolithin or an urolithin or a pharmaceutically acceptable salt thereof improves the muscle bioenergetics.

In a seventeenth aspect, the present disclosure provides a method for increasing the level of phosphocreatine in a muscle of a subject in need thereof, the method comprising administering to the subject in need thereof an effective amount of a composition comprising urolithin or an urolithin or a pharmaceutically acceptable salt thereof. In other words, the present disclosure provides a composition comprising urolithin or an urolithin or a pharmaceutically acceptable salt thereof for use in a method for increasing the level of phosphocreatine in a muscle of a subject in need thereof.

Phosphocreatine serves as a rapidly mobilizable reserve of high-energy phosphates. Thus, in one embodiment administering to the subject in need thereof an effective amount of a composition comprising urolithin or an urolithin or a pharmaceutically acceptable salt thereof improves the energy utilization of the muscle.

In one embodiment, the methods disclosed herein can also be effective in the treatment of a disease or condition selected from chronic kidney disease, metabolic induced muscle wasting, end-stage renal disease, dialysis, diabetes, muscle loss and/or kidney failure due to hospitalization in the intensive care unit, Pompe disease, metabolic acidosis, methylmalonic aciduria, disuse atrophy, protein-energy wasting and combinations thereof.

In another embodiment the disease or condition is selected from the group consisting of chronic kidney disease, metabolic induced muscle wasting, end-stage renal disease, dialysis, diabetes, muscle loss and/or kidney failure due to hospitalization in the intensive care unit, Pompe disease, metabolic acidosis, methylmalonic aciduria, disuse atrophy, protein-energy wasting. Preferably the disease is chronic kidney disease.

In a further embodiment, the methods disclosed herein can also be effective in the treatment of an early stage of chronic kidney disease and/or a late stage of chronic kidney disease. Preferably, the early stage of chronic kidney disease are stages 2 and 3. In a certain embodiment the early stage of chronic kidney disease is stage 2. Preferably, the late stage of chronic kidney disease are stages 4 and 5. In a certain embodiment the late stage of chronic kidney disease is stage 4.

In one embodiment, the methods disclosed herein can also be effective in the treatment of an early stage of chronic kidney disease.

In another embodiment, the methods disclosed herein can also be effective in the treatment of a late stage of chronic kidney disease.

In one embodiment, the amount of the composition comprising urolithin or the urolithin or a pharmaceutically acceptable salt thereof is effective to increase the urinary creatinine and/or to decrease the albumin/creatinine ratio and/or to increase the albumin reabsorption and/or to improve the lean mass of a muscle and/or to increase the urinary carnosine and/or to increase the urinary anserine and/or to increase urinary S-adenosylmethionine and/or to improve the bone femur mass.

In a certain embodiment, the amount of the composition comprising urolithin or the urolithin or a pharmaceutically acceptable salt thereof is effective to increase the urinary creatinine and to improve the lean mass of a muscle.

In another embodiment, the amount of the composition comprising urolithin or the urolithin or a pharmaceutically acceptable salt thereof is effective to improve the lean mass of a muscle and/or to improve the muscle fiber size and/or to improve the level of at least one amino acid.

In a further embodiment the amount of the composition comprising urolithin or the urolithin or a pharmaceutically acceptable salt thereof is effective to improve the level of at least one metabolic product and/or to improve the level of at least one nucleotide and/or to improve the level of at least one nicotinamide adenine dinucleotide and/or to increase the level of glutathione (GSH) and/or glutathione disulfide (GSSG) and/or to increase the level of succinate and/or malate and/or to increase the level of phosphocreatine.

In one embodiment, the composition comprising urolithin or the urolithin or a pharmaceutically acceptable salt thereof of the methods disclosed herein is administered in a composition selected from the group consisting of a food product, a food for special medical purposes (FSMP), a nutritional supplement, a dairy-based drink, a low-volume liquid supplement, a meal replacement beverage and combinations thereof. In particular, the composition may comprise additional supplements, such as minerals, vitamins and further bioactive substances such as N-acetylglucosamine or N-acetylmuramic acid.

Accordingly, in an eighteenth aspect, the present disclosure provides a nutritional composition for use in treating and/or preventing a disease or condition associated with muscle decline and/or a kidney dysfunction. The nutritional composition comprises a composition comprising urolithin or an urolithin or a pharmaceutically acceptable salt thereof.

In one embodiment the nutritional composition contains an amount of urolithin effective for treating and/or preventing a disease or condition associated with muscle decline and/or a kidney dysfunction in a subject in need thereof. In a related embodiment, the nutritional composition is formulated for oral administration.

In a nineteenth aspect, the present disclosure provides a unit dosage form for use in treating and/or preventing a disease or condition associated with muscle decline and/or a kidney dysfunction. The unit dosage form comprises a composition comprising urolithin or an urolithin or a pharmaceutically acceptable salt thereof.

In one embodiment the unit dosage form contains an amount of urolithin effective for treating and/or preventing a disease or condition associated with muscle decline and/or a kidney dysfunction in a subject in need thereof. In a related embodiment, the unit dosage form is formulated for enteral administration.

In one embodiment, the amount of the nutritional composition and/or the unit dosage form is effective to increase the urinary creatinine and/or to decrease the albumin/creatinine ratio and/or to increase the albumin reabsorption and/or to improve the lean mass of a muscle and/or to increase the urinary carnosine and/or to increase the urinary anserine and/or to increase urinary S-adenosylmethionine and/or to improve the bone femur mass.

In another embodiment, the amount of the nutritional composition and/or the unit dosage form is effective to improve the lean mass of a muscle and/or to improve the muscle fiber size and/or to improve the level of at least one amino acid.

In a further embodiment the amount of the nutritional composition and/or the unit dosage form is effective to improve the level of at least one metabolic product and/or to improve the level of at least one nucleotide and/or to improve the level of at least one nicotinamide adenine dinucleotide and/or to increase the level of glutathione (GSH) and/or glutathione disulfide (GSSG) and/or to increase the level of succinate and/or malate and/or to increase the level of phosphocreatine.

In a certain embodiment, the amount of the nutritional composition and/or the unit dosage form is effective to increase the urinary creatinine and to improve the lean mass of a muscle.

In an embodiment, the nutritional composition and/or the unit dosage form is selected from the group consisting of a food product, a food for special medical purposes (FSMP), a nutritional supplement, a dairy-based drink, a low-volume liquid supplement, a meal replacement beverage and combinations thereof.

Food products according to the present invention include, but are not limited to, breads, cakes, cookies, crackers, extruded snacks, potato products, rice products, corn products, wheat products, dairy products, yogurt, confectionery, hard candy, gummy candies, nutrition bar, breakfast cereal or beverage. A Food product according to the present invention may also be a plant-based drink such as a juice, a smoothie, soy milk, rice milk, or almond milk.

In a further embodiment, the nutritional composition and the unit dosage form disclosed herein can be administered to a subject in an early stage of chronic kidney disease and/or a late stage of chronic kidney disease.

In another embodiment, the nutritional composition and the unit dosage form disclosed herein can be administered to a subject in an early stage of chronic kidney disease.

In one embodiment, the nutritional composition and the unit dosage form disclosed herein can be administered to a subject in a late stage of chronic kidney disease.

In another embodiment, urolithin is administered in form a composition comprising urolithin.

In a further embodiment, urolithin in pure form or a pharmaceutically acceptable salt thereof is administered.

In one embodiment, the compositions disclosed herein further comprise at least one additive.

In another embodiment, urolithin is micronized for more rapid dispersion or dissolution. If micronized urolithin is used, then preferably the D₅₀ is under 100 μm, i.e., 50% by mass of the urolithin or precursor thereof has a particle diameter size under 100 μm. More preferably, the urolithin or precursor thereof has a D₅₀ of under 75 μm, for example under 50 μm, for example under 25 μm, for example under 20 μm, for example under 10 μm. More preferably, the urolithin or precursor thereof has a D₅₀ in the range 0.5 to 50 μm, for example 0.5 to 20 μm, for example 0.5 to 10 μm, for example 1.0 to 10 μm, for example 1.5 to 7.5 μm, for example 2.8 to 5.5 μm. Preferably, the urolithin or precursor thereof has a D₉₀ size under 100 μm. More preferably, the urolithin or precursor thereof has a D₉₀ size under 75 μm, for example under 50 μm, for example under 25 μm, for example under 20 μm, for example under 15 μm. The urolithin or precursor thereof preferably has a D₉₀ in the range 5 to 100 μm, for example 5 to 50 μm, for example 5 to 20 μm, for example 7.5 to 15 μm, for example 8.2 to 16.0 μm.

Preferably, the urolithin has a D₁₀ in the range 0.5-1.0 μm. Preferably, the urolithin has a D₉₀ in the range 8.2 to 16.0 μm, a D₅₀ in the range 2.8 to 5.5 μm and a D₁₀ in the range 0.5 to 1.0 μm.

Miconization can be achieved by a method selected from the group consisting of compressive force milling, hammermilling, universal or pin milling, and jet milling such as spiral jet milling or fluidized-bed jet milling. Jet milling is particularly preferred.

Urolithins are metabolites of dietary ellagic acid derivatives, such as ellagitannins, and are produced in the human gut by gut bacteria.

Ellagitannins are a class of antioxidant polyphenols found in several fruits, particularly pomegranate, strawberries, raspberries and walnuts. Although the absorption of ellagitannins is extremely low, they are rapidly metabolized by the gut microbiota of the large intestine into urolithins.

Due to their superior absorption, urolithins are believed to be the bioactive molecules mediating the effects of ellagitannins. To that end, for example, urolithins were previously shown to have anti-proliferative, antioxidant and anti-inflammatory properties.

Example urolithins include ellagic acid, urolithin A (3,8-dihydroxyurolithin), urolithin B (3-hydroxyurolithin), urolithin C (3,8,9-trihydroxyurolithin), urolithin D (3,4,8,9-tetrahydroxyurolithin), urolithin A glucuronide and urolithin B glucuronide. Ellagic acid and Urolithins A, B, C and D have the following structure:

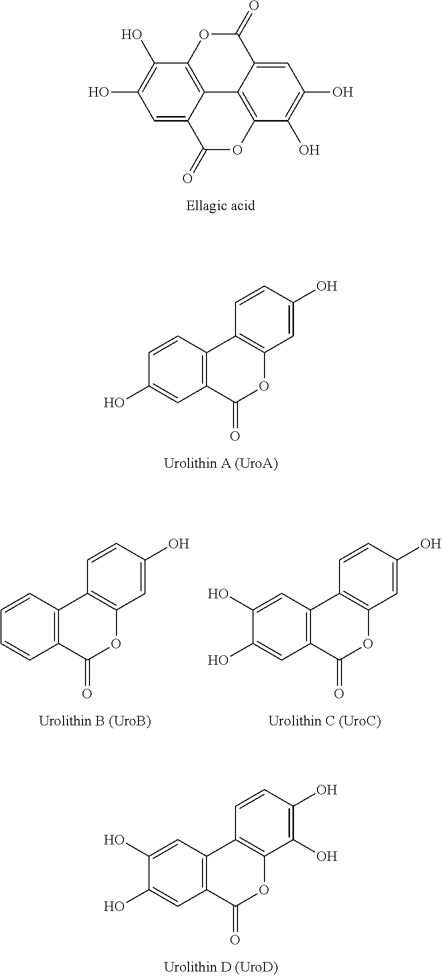 [SMILES: O=c1oc2c(O)c(O)cc3c(=O)oc4c(O)c(O)cc1c4c23 ; O=c1oc2cc(O)ccc2c2ccc(O)cc12 ; O=c1oc2cc(O)ccc2c2ccccc12 ; O=c1oc2cc(O)ccc2c2cc(O)c(O)cc12 ; O=c1oc2c(O)c(O)ccc2c2cc(O)c(O)cc12]

The inventive composition may comprise urolithins as defined above, extracted from or comprised in several fruits, such as pomegranate extract, tamarind extract or mumijo extract, strawberries, raspberries and walnuts, that typically provide at least a portion of the at least one urolithin or precursor thereof. Generally, inventive composition may comprise either such a pomegranate extract, tamarind extract or mumijo extract, strawberries, raspberries and walnuts, or more preferably urolithins extracted from such a pomegranate extract, tamarind extract or mumijo extract, strawberries, raspberries and walnuts. Alternatively, or additionally, the inventive composition may comprise any of the urolithins as defined herein in isolated form. An isolated form may be prepared either on basis of natural sources, such as the sources identified above, or may be provided by chemical synthesis.

In one embodiment, the urolithin is selected from the group consisting of urolithin A, urolithin B, urolithin C, urolithin D, glucuronated forms thereof, methylated forms thereof, sulfated forms thereof, and mixtures thereof. The urolithin can be provided as an isolated urolithin, e.g., isolated from a natural source or prepared by total synthesis. Isolated urolithins may be synthesized de novo.

In a certain embodiment, the urolithin is urolithin A.

Urolithin can be administered in an amount of about 0.2-150 milligram (mg) of urolithin per kilogram (kg) of body weight of the subject. Preferably, the urolithin is administered in a dose equal or equivalent to 2-120 mg of urolithin per kg body weight of the subject, more preferably 4-90 mg of urolithin per kg body weight of the subject, particular preferably 6-20 mg of urolithin per kg body weight of the subject. most preferably 16 mg of urolithin per kg body weight of the subject.

In an embodiment, the urolithin is administered in a dose sufficient to achieve a peak serum level of at least 0.001 micromolar (μM), preferably at least 0.01 μM, more preferably at least 0.1 μM, most preferably at least 1 μM, at least 5 μM or at least 10 μM. In an embodiment, the urolithin or precursor thereof is administered in a dose sufficient to achieve a sustained serum level of at least 0.001 micromolar (μM), preferably at least 0.01 μM, more preferably at least 0.1 μM, most preferably at least 1 μM, at least 5 μM or at least 10 μM. The sustained serum level can be measured using any suitable method, for example, high pressure liquid chromatography (HPLC) or HPLC-MS.

In some embodiments, the urolithin is 0.1 to 80 wt. % of the composition, for example 0.1 to 60 wt. % of the composition, such as 0.25 to 50 wt. % of the composition, 0.5-50 wt. % of the composition. If the composition is provided as part of or the whole of a meal, then the urolithin can be 0.25-5 wt. % of the composition, for example 0.3-3 wt. % of the composition. If the composition is provided as a single serving supplement to a subject's general diet, then the urolithin can be 20 to 80 wt. % w/w of the composition, for example 20 to 40 wt. % of the composition, for example 25 to 35 wt. % of the composition.

The urolithin can be administered in an amount of about 12 mg/day to about 9 g/day, preferably about 12 mg/day to about 7 g/day, more preferably about 12 mg/day to about 5 g/day, most preferably about 12 mg/day to about 3 g/day, for example about 12 mg/day to about 900 mg/day, about 12 mg/day to about 700 mg/day, about 12 mg/day to about 500 mg/day, about 12 mg/day to about 250 mg/day, about 12 mg/day to about 100 mg/day, or about 12 mg/day to about 50 mg/day, or about 12 mg/day to about 20 mg/day, or about 12 mg/day to about 18 mg/day. Of course, the daily dose can be administered in portions at various hours of the day. However, in any given case, the amount of compound and/or composition administered will depend on such factors as the solubility of the active component, the formulation used, subject condition (such as weight), and/or the route of administration. For example, the daily doses of urolithin disclosed above are non-limiting and, in some embodiments, may be different; in particular, the composition comprising urolithin or urolithin or a pharmaceutically acceptable salt thereof as disclosed herein can be utilized as an acute care food for special medical purposes (FSMP) and contain up to about 100 mg urolithin/day.

In one embodiment, Administration of the composition comprising urolithin or the urolithin or a pharmaceutically acceptable salt thereof may be carried out for at least about 2 or 3 months, preferably at least about 4 or 5 months, more preferably at least about 6 or 7 months in the remission phase, such as about 2 to 60 months, 2 to 48 months, 2 to 36 months, 2 to 24 months, or 2 to 12 months, preferably such as about 4 to 60 months, 4 to 48 months, 4 to 36 months, 4 to 24 months, or 4 to 12 months, etc.

In another embodiment, a composition comprising urolithin or an urolithin or a pharmaceutically acceptable salt thereof is administered for about 1-2 weeks to 6 months, more preferably about 2 weeks to 4 months, more preferably about 3 weeks to 3 weeks, and most preferably about 4 weeks to 10 weeks to increase the urinary creatinine and/or to decrease the albumin/creatinine ratio and/or to increase the albumin reabsorption and/or to improve the lean mass of a muscle and/or to increase the urinary carnosine and/or to increase the urinary anserine and/or to increase urinary S-adenosylmethionine and/or to improve the bone femur mass.

In a further embodiment, a composition comprising urolithin or an urolithin or a pharmaceutically acceptable salt thereof is administered for 5 weeks to obtain an increase of the urinary creatinine and/or a decrease of the albumin/creatinine ratio and/or an increase of the albumin reabsorption and/or an improvement of the lean mass of a muscle.

In one embodiment, a composition comprising urolithin or an urolithin or a pharmaceutically acceptable salt thereof is administered for 10 weeks to obtain an increase of the urinary creatinine and/or a decrease of the albumin/creatinine ratio and/or an increase of the albumin reabsorption and/or an improvement of the lean mass of a muscle and/or an increase of the urinary carnosine and/or an increase of the urinary anserine and/or an increase of the urinary S-adenosylmethionine and/or an improvement the bone femur mass.

In another embodiment, the amount of the composition comprising urolithin or the urolithin or a pharmaceutically acceptable salt thereof is administered for 10 weeks to improve the lean mass of a muscle and/or to improve the muscle fiber size and/or to improve the level of at least one amino acid.

In a further embodiment the amount of the composition comprising urolithin or the urolithin or a pharmaceutically acceptable salt thereof is administered for 10 weeks to improve the level of at least one metabolic product and/or to improve the level of at least one nucleotide and/or to improve the level of at least one nicotinamide adenine dinucleotide and/or to increase the level of glutathione (GSH) and/or glutathione disulfide (GSSG) and/or to increase the level of succinate and/or malate and/or to increase the level of phosphocreatine.

The composition comprising urolithin or an urolithin or a pharmaceutically acceptable salt thereof as disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms, such as tablets, capsules, powders, granules, ointments, solutions, suppositories, injections, inhalants, gels, microspheres, and aerosols. As such, administration of the composition comprising urolithin or urolithin or a pharmaceutically acceptable salt thereof can be achieved in various ways, including oral, buccal, rectal, parenteral, intraperitoneal, intradermal, transdermal, and intratracheal administration. The active agent urolithin may be systemic after administration or may be localized by the use of regional administration, intramural administration, or use of an implant that acts to retain the active dose at the site of implantation.

In one embodiment, the composition comprising urolithin or urolithin or a pharmaceutically acceptable salt thereof is administered enterally.

For oral preparations, the composition comprising urolithin or urolithin or a pharmaceutically acceptable salt thereof can be used alone or in combination with appropriate additives to make tablets, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The composition comprising urolithin or urolithin or a pharmaceutically acceptable salt thereof can be utilized in an aerosol formulation to be administered by inhalation. For example, the composition comprising urolithin or urolithin or a pharmaceutically acceptable salt thereof can be formulated into pressurized acceptable propellants such as dichlorodifluoromethane, propane, nitrogen and the like.

Furthermore, the composition comprising urolithin or urolithin or a pharmaceutically acceptable salt thereof can be made into suppositories by mixing with a variety of bases such as emulsifying bases or water-soluble bases. The composition comprising urolithin or urolithin or a pharmaceutically acceptable salt thereof can be administered rectally by a suppository. The suppository can include a vehicle such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition comprising urolithin or urolithin or a pharmaceutically acceptable salt thereof.

In another embodiment, the composition comprising urolithin or urolithin or a pharmaceutically acceptable salt thereof is administered parenterally. Non-limiting examples of parenteral administration include intravenously, intramuscularly, intraperitoneally, subcutaneously, intraarticularly, intrasynovially, intraocularly, intrathecally, topically, and inhalation. As such, non-limiting examples of the form of the composition comprising urolithin or urolithin or a pharmaceutically acceptable salt thereof include natural foods, processed foods, natural juices, concentrates and extracts, injectable solutions, microcapsules, nano-capsules, liposomes, plasters, inhalation forms, nose sprays, nosedrops, eyedrops, sublingual tablets, and sustained-release preparations.

Unit dosage forms for injection or intravenous administration may comprise the composition comprising urolithin or urolithin or a pharmaceutically acceptable salt thereof in a composition as a solution in sterile water, normal saline or another pharmaceutically acceptable carrier, wherein each dosage unit, for example, mL or L, contains a predetermined amount of the composition containing one or more of the composition comprising urolithin or urolithin or a pharmaceutically acceptable salt thereof.

### EXAMPLES

### Example 1

The following non-limiting example discusses experimental data that are investigated to further support the methods and compositions disclosed herein.

The renoprotective effects of Urolithin A were assessed in a mouse model of type 2 diabetes (the db/db mouse).

A mouse model of diabetes was used endorsed by the Diabetic Complications Consortium (DiabComp/AMDCC), the db/db mouse model, which is obese and insulin resistant “type 2”. This mouse model has been shown to have impaired mitophagy in the kidney.

Mice were purchased from The Jackson Laboratory (Stock No: 000642) in order to obtain sizeable cohorts to run groups in tandem, which is crucial for reliable testing of mitochondrial function. Eight to nine week-old male db/db mice (n=15 per group) received Urolithin A by oral gavage (50 mg/kg/day) for a duration of 10 weeks. Db/m (control) or db/db (diabetic) mice were randomized to the following treatments:

TABLE 1
Experimental group overview.

| Group | Mice | Treatment | Dose | Number (n) |
|---|---|---|---|---|
| 1 | Db/m | Vehicle (0.5% CMC) | — | 15 |
| 2 | Db/db | Vehicle (0.5% CMC) | — | 15 |
| 3 | Db/db | Urolithin A | 50 mg/kg/day | 15 |

Animals used were 15 db/m and 30 db/db procured from Jackson Laboratory. The mice had free access to tap water and to standard rodent chow diet (Specialty Feeds, WA, Australia). All mice were kept in a room with constant temperature of ˜25° C. with 12 h day/night cycle.

A suspension of 0.5% carboxymethylcellulose (CMC; Sigma #419303) was first prepared. Urolithin A (16.67 mg/ml) was prepared by dissolving in 0.5% CMC solution and left stirring at room temperature. Urolithin A was dosed at 50 mg/kg/day. The weights of the mice were used to calculate the dosage of Urolithin A to be administered to each mouse, based on weekly bodyweight measurements. Approximately 1001 of 0.5% CMC was given to mice daily in the vehicle groups.

At baseline, mid-point and one week prior to euthanasia, mice underwent metabolic caging for 24 hours to collect urine and to measure food and water intake. Body composition by EchoMRI was performed at baseline, mid-point and two weeks prior to euthanasia to determine fat mass and lean body mass. At baseline and week 9, an oral glucose tolerance test was performed to assess glucose handling. Fasting plasma glucose was measured using a glucose colorimetric assay kit (Cayman, Ann Arbor, USA) at baseline, 15 min and 60 min after glucose treatment. Blood pressure measurements were performed at week 10.

All analyses were performed by ANOVA followed by post hoc paired analysis using Tukey's least significant difference method, correcting for multiple comparisons. A value for p<0.05 was considered as statistically significant. A Z-score of 2 was used to exclude outliers.

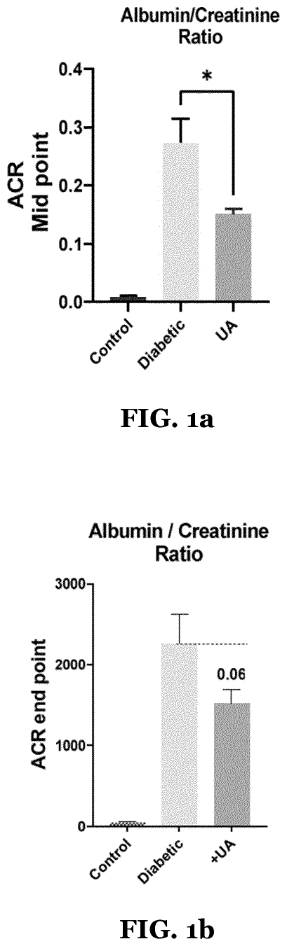

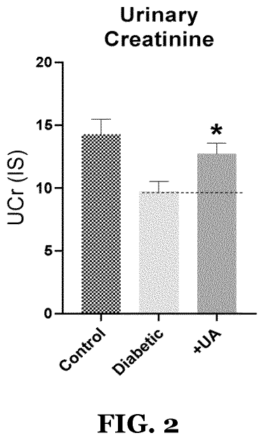

One week prior to cull, 24-hour metabolic caging was performed to collect urine, and blood was collected from the submandibular vein. Urine and plasma creatinine was determined using the Cobas Integra autoanalyzer. Urine albumin was measured by ELISA (Bethyl, USA). Blood pressure was determined by tail cuff plethysmography. As a result, the albumin/creatinine ratio was significantly decreased in db/db+UA group (UA) when compared to the db/db+V group (Diabetic) after 5 weeks of treatment (FIG. 1a) and after 10 weeks of treatment (FIG. 1b). In addition, the results show that the urinary creatinine in UA-treated db/db mice was higher compared to the vehicle-treated group (FIG. 2).

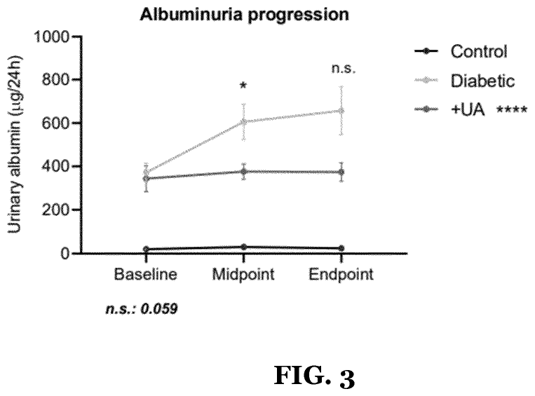

Excretion of albumin in the urine over a 24-hour period was measured by ELISA kit (Bethyl). At the endpoint of the study, there was a significant increase in albuminuria in db/db mice when compared to db/m mice. Treatment of UA did affect albuminuria in db/db mice at midpoint. Creatinine clearance was calculated from urine and plasma creatinine measured using the Creatinine plus ver.2 (CREP2) assay on a Cobas Integra 400 plus autoanalyzer (Roche Diagnostics). There was a decline in kidney function in db/db mice (Diabetic) as measured by an increase in albuminuria and reduced creatinine clearance at 18-19 weeks of age. Treatment of UA delayed the progression of albuminuria in the db/db mice. This result indicates that the progression of albuminuria in UA-treated db/db (UA) mice was significantly delayed when compared to their vehicle-treated counterparts (FIG. 3).

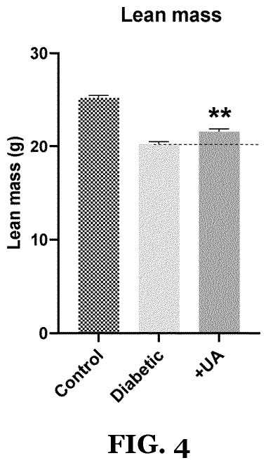

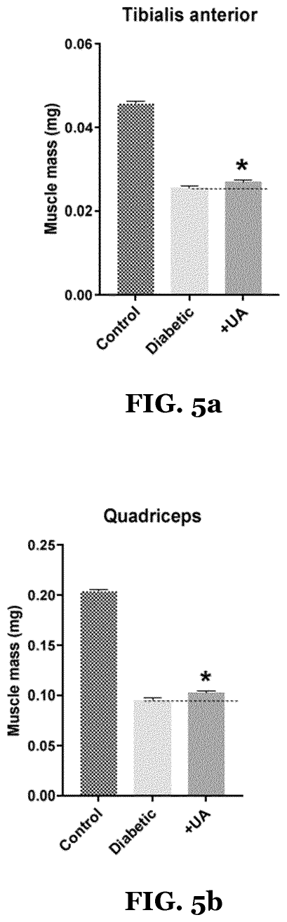

Whole body composition including lean mass and fat mass were measured using EchoMRI at baseline, midpoint and endpoint. Overall, db/db mice (Diabetic) had an increase in fat mass and total body mass, and a decrease in lean mass when compared to db/m (Control), irrespective of treatment. Lean mass was significantly increased in db/db+UA (UA) group when compared to the db/db+V group (Diabetic). The results are shown in FIG. 4. In particular, the tibialis anterior and the quadriceps of the db/db+UA (UA) group show a significantly increased muscle mass compared to the db/db+V group (Diabetic). The results are shown in FIG. 5a and FIG. 5b.

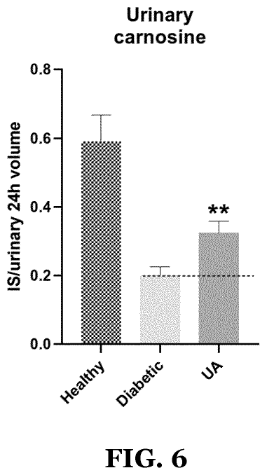

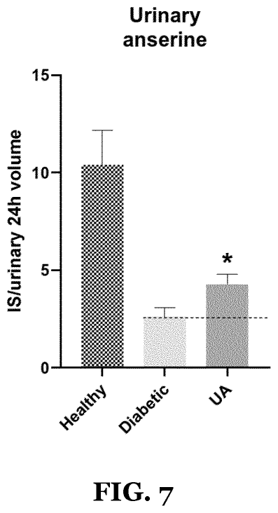

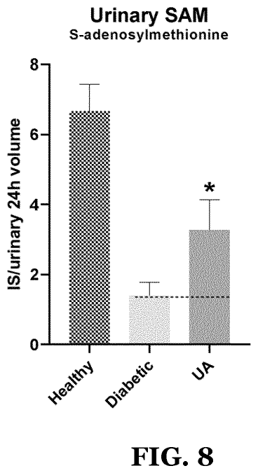

Urinary levels of carnosine, anserine and S-adenosylmethionine (SAM) were measured in a semi-quantitative manner using HILIC chromatography and ESI-orbitrap mass spectrometry. The levels of all these metabolites were decreased in the db/db+V group (Diabetic) compared to the db/m group (Healthy) (FIG. 6-8). The levels of urinary carnosine were significantly increased in the db/db+UA (UA) group when compared to the db/db+V group (Diabetic). The results are shown in FIG. 6. The levels of urinary anserine were significantly increased in the db/db+UA (UA) group when compared to the db/db+V group (Diabetic). The results are shown in FIG. 7. The levels of urinary S-adenosylmethionine (SAM) were significantly increased in the db/db+UA (UA) group when compared to the db/db+V group (Diabetic). The results are shown in FIG. 8.

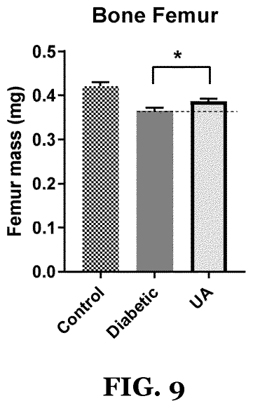

Left and right femur bones were collected at the end of study, after 10 week treatment, and both femurs were cleaned and snap frozen, weighed and stored at −80° C. Femur bone mass was decreased in db/db+V mice compared to db/m group (Control), but it is significantly increased in the db/db+UA group (UA) compared to the db/db+V group (Diabetic). The results are shown in FIG. 9.

The fiber size distribution measured on tibilais interior in mice after 10 weeks treatment with Urolithin A, dosed at 50 mg/kg/day (+UA, db/db+UA group) compared to a vehicle group (Diabetic, db/db+V group) and a control group (Control, db/m+V group) after 10 weeks treatment with 100 μl of 0.5% carboxymethylcellulose.

Tibialis anterior, EDL, gatrocnemius, soleus and quadriceps muscle from Monash study were harvested, weighted and cryo conserved.

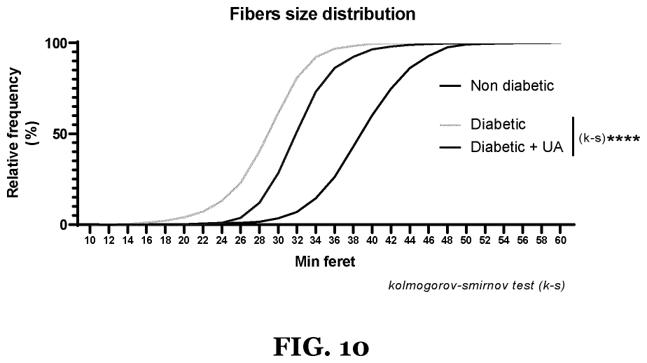

The fiber size distribution was determined after tibialis anterior cryosection, stained for the laminin protein and the myo-nucleus. All slides were acquired with the Olympus VS120 slide scanner microscope. The size of myofibers was calculated with Min Feret using an automated image processing algorithm developed internally using QuPath software and Fiji's tool open-CSAM. The results are shown in FIG. 10.

The metabolites in muscle tissue were determined in mice after 10 weeks treatment with Urolithin A, dosed at 50 mg/kg/day (+UA, db/db+UA group) compared to a vehicle group (Diabetic, db/db+V group) and a control group (Healthy, db/m+V group) after 10 weeks treatment with 100 μl of 0.5% carboxymethylcellulose.

The extraction of the muscle tissue was done with metal beads in pre-cooled racks (−80° C.) for 2 min at 23 Hz in a tissue mixer (Qiagen TissueLyser II).

All samples were agitated for 10 minutes at 1500 rpm and 4° C. in a shaker (Thermomixer C, Eppendorf), followed by centrifugation for 10 minutes and 15,000 rpm at 4° C. Notably, for experiments, which did not involve an isotopically labelled substrate, labelled internal standards (fully labelled ¹³C yeast extract and nicotinamide-D4) were included during extraction for later data normalization. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layers of muscle samples were quantified with a bicinchoninic acid (BCA) assay (ThermoFisher Scientific) and used for later normalization of the metabolite concentrations.

2 μL of each sample were injected into a hydrophilic interaction chromatography (HILIC) analytical column (2.1 mm×150 mm, 5 μm pore size, 200 Å HILICON iHILIC®-Fusion(P)), guarded by a pre-column (2.1 mm×20 mm, 200 Å HILICON iHILIC®-Fusion(P) Guard Kit) operating at 35° C. The separation was achieved by applying a linear solvent gradient. As mobile phases, solvent A was H2O with 10 mM ammonium acetate (NH4Ac) and 0.04% (v/v) ammonium hydroxide (NH4OH), pH ˜9.3, and solvent B was acetonitrile (ACN).

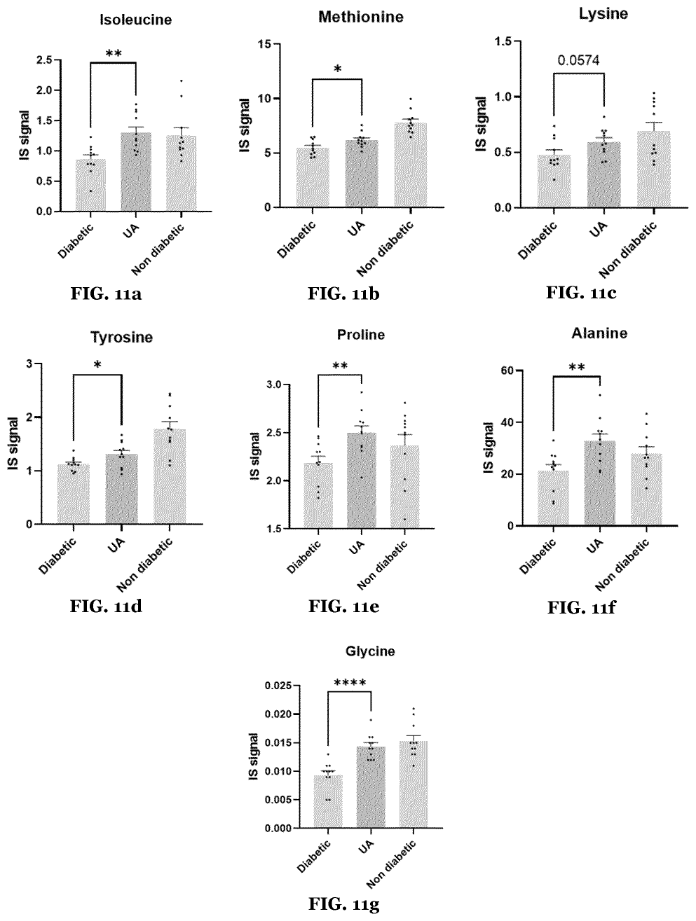

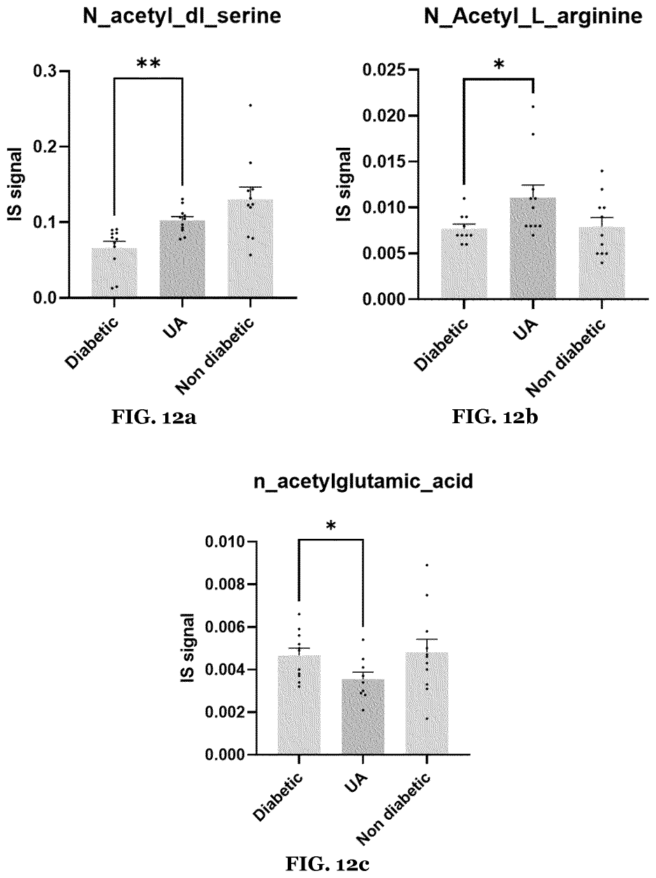

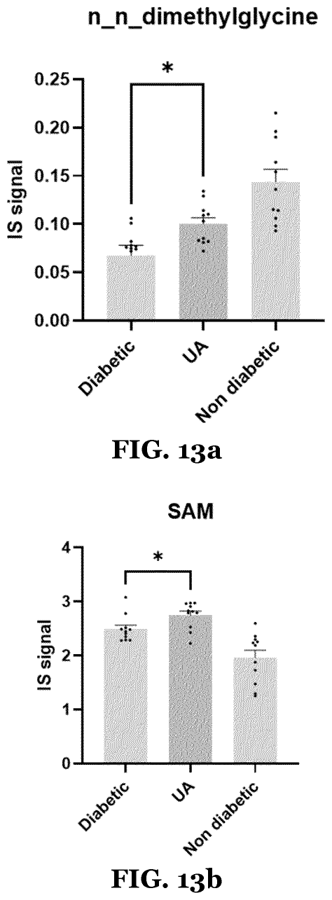

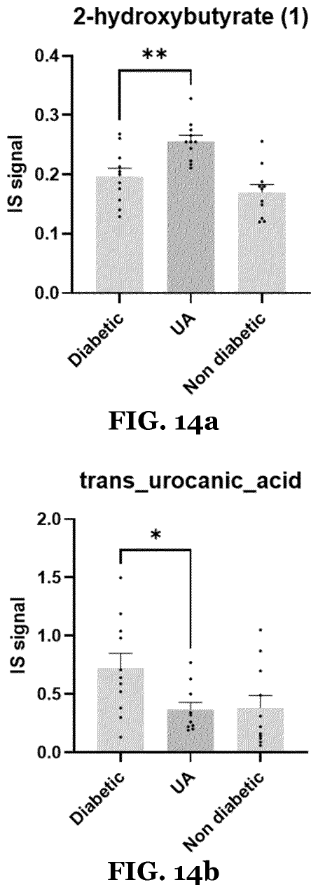

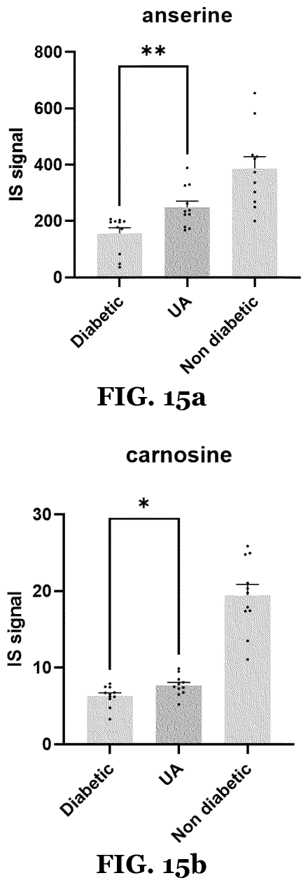

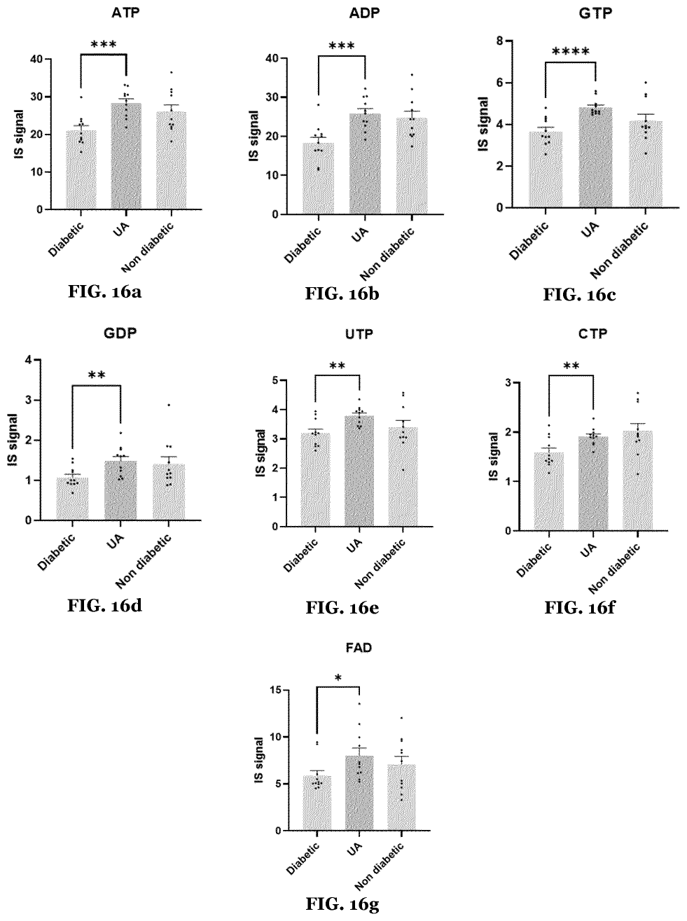

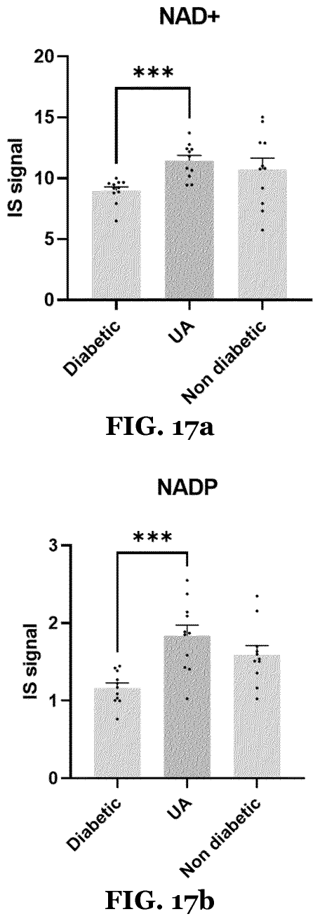

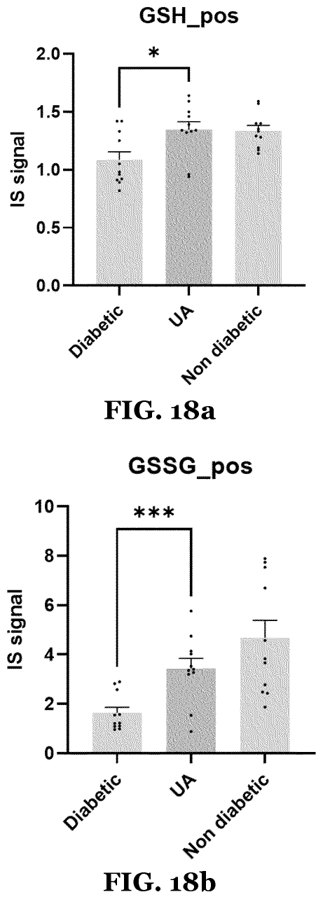

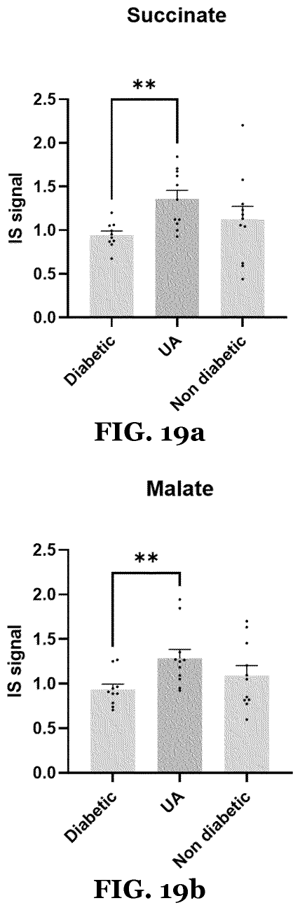

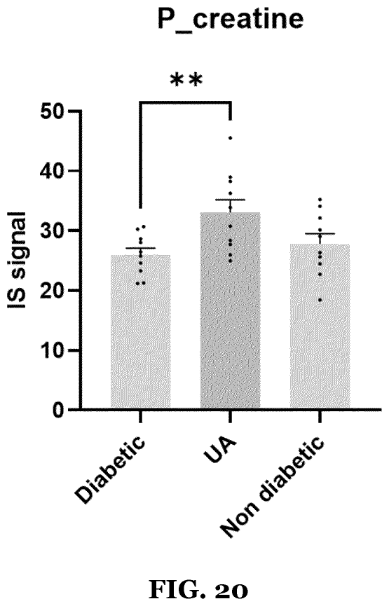

The sheath gas was 20 AU, and the auxiliary gas was kept 15 AU. The temperature of vaporizer was 280° C. and the temperature of the ion transfer tube was 310° C. The full scan was measured with on-the-fly alternating positive and negative mode scans, which covered m/z ranges from 83 to 830 and from 73 to 900, respectively, at a resolution of 60′000. The eluting metabolites, were analyzed with an orbitrap mass spectrometer (Orbitrap Fusion Lumos Tribrid, Thermo Scientific) equipped with a heated electrospray ionization (H-ESI) source. The software Xcalibur v4.1.31.9 (Thermo Scientific) was used for instrument control and data processing of isotopically labelled and unlabelled metabolites. The results for isoleucine, methionine, lysine, tyrosine, proline, alanine, glycine, N-acetyl-DL-serine, N-acetyl-L-arginine, N-acetylglutamic acid, N,N-dimethylglycine, SAM, 2-hydroxybutyrate, trans-urocanic acid, anserine, carnosine, ATP, ADP, GTP, GDP, UTP, CTP, FAD, NAD+, NADP+, GSH, GSSG, succinate, malate and phosphocreatine are shown in FIG. 11-20.

1. A method of treating and/or preventing a disease or condition associated with muscle decline and/or a kidney dysfunction, the method comprising administering to a subject in need thereof an effective amount of a composition comprising urolithin or an urolithin or a pharmaceutically acceptable salt thereof.

2. The method of claim 1, wherein the disease or condition is selected from the group consisting of chronic kidney disease, metabolic induced muscle wasting, end-stage renal disease, dialysis, diabetes, muscle loss and/or kidney failure due to hospitalization in the intensive care unit, Pompe disease, metabolic acidosis, methylmalonic aciduria, disuse atrophy, protein-energy wasting and combinations thereof.

3. The method of claim 1, wherein the muscle decline and/or the kidney dysfunction is treated and/or prevented in an early stage of chronic kidney disease and/or a late stage of chronic kidney disease.

4. (canceled)

5. The method of claim 1, wherein the composition comprising urolithin or urolithin or a pharmaceutically acceptable salt thereof is administered enterally.

6. (canceled)

7. The method of claim 1, wherein the composition comprising urolithin or the urolithin or a pharmaceutically acceptable salt thereof is administered in a composition selected from the group consisting of a food product, a food for special medical purposes (FSMP), a nutritional supplement, a dairy-based drink, a low-volume liquid supplement, a meal replacement beverage, and combinations thereof.

8-9. (canceled)

10. A method for improving the lean mass of a muscle in a subject in need thereof, the method comprising administering to the subject in need thereof an effective amount of a composition comprising urolithin or an urolithin or a pharmaceutically acceptable salt thereof.

11. The method of claim 10, wherein the subject has a disease or condition selected from the group consisting of chronic kidney disease, metabolic induced muscle wasting, end-stage renal disease, dialysis, diabetes, muscle loss and/or kidney failure due to hospitalization in the intensive care unit, Pompe disease, metabolic acidosis, methylmalonic aciduria, disuse atrophy, protein-energy wasting and combinations thereof.

12-13. (canceled)

14. The method of claim 10, wherein the composition comprising urolithin or urolithin or a pharmaceutically acceptable salt thereof is administered enterally.

15-18. (canceled)

19. A method for increasing the albumin reabsorption in a subject in need thereof, the method comprising administering to the subject in need thereof an effective amount of a composition comprising urolithin or an urolithin or a pharmaceutically acceptable salt thereof.

20. The method of claim 19, wherein the subject has a disease or condition selected from the group consisting of chronic kidney disease, metabolic induced muscle wasting, end-stage renal disease, dialysis, diabetes, muscle loss and/or kidney failure due to hospitalization in the intensive care unit, Pompe disease, metabolic acidosis, methylmalonic aciduria, disuse atrophy, protein-energy wasting and combinations thereof.

21. The method of claim 19, wherein the albumin reabsorption is increased in an early stage of chronic kidney disease and/or a late stage of chronic kidney disease.

22. The method of claim 19, wherein the composition comprising urolithin or urolithin or a pharmaceutically acceptable salt thereof is administered enterally.

23. (canceled)

24. The method of claim 19, wherein the composition comprising urolithin or the urolithin or a pharmaceutically acceptable salt thereof is administered in a composition selected from the group consisting of a food product, a food for special medical purposes (FSMP), a nutritional supplement, a dairy-based drink, a low-volume liquid supplement, a meal replacement beverage and combinations thereof.

25. The method of claim 19, wherein the urolithin is micronized.

26. The method of claim 19, wherein the urolithin is urolithin A.

27-160. (canceled)
